(19) United States
(12) Patent Application Publication
(10) Pub. No.: US 2025/0389703 A1
(43) Pub. Date: Dec. 25, 2025

(54) ARRANGEMENT FOR ALIGNING A NEEDLE AND A DRY STICK WHEN APPLYING A MILK SAMPLE OF AN ANIMAL

(71) Applicant: DELAVAL HOLDING AB, Tumba (SE)

(72) Inventors: Thomas Nikolai CARLSEN, Tumba (SE); Kristjan Freyr GUDMUNDSSON, Tumba (SE); Ehsan NOROUZI, Tumba (SE)

(21) Appl. No.: 18/879,742

(22) PCT Filed: Jun. 21, 2023
(86) PCT No.: PCT/SE2023/050643

§ 371 Date: Dec. 27, 2024

(30) Foreign Application Priority Data
Jun. 29, 2022 (SE) 2250808-9

(51) Int. Cl. G01N 33/04; A01J 5/013; G01N 21/77; G01N 21/78

(52) U.S. Cl. CPC G01N 33/04; A01J 5/0131; G01N 21/78; G01N 2021/7759

(57) ABSTRACT

An arrangement for aligning a needle and dry stick when applying an animal milk sample. The dry stick indicates a biomarker value of the milk sample. The arrangement comprises a carrier supporting the dry stick, a tube element for receiving the milk sample, and a needle connected to the tube element. A light source illuminates the dry stick, while a camera captures images of the dry stick and its shadow. A drive unit moves the carrier with the dry stick, controlled by a controller. The controller triggers the camera to capture and analyze images, using the shadow analysis to precisely position the dry stick. Based on this image analysis, the controller signals the drive unit to move the dry stick into proper alignment, enabling accurate application of the milk sample by the needle.

### TECHNICAL FIELD

This document discloses an arrangement. More particularly, an arrangement is described, arranged to align a needle and a dry stick in order to apply a milk sample of an animal to the dry stick when the dry stick is positioned in a milk sample apply position in which the needle is aligned with the dry stick, wherein the dry stick is configured to indicate at least one biomarker value of the applied milk sample.

### BACKGROUND

On an animal farm, it is important to keep the animals healthy in order to enhance milk/meat production. On a dairy farm, for example, it is very important to inseminate animals at an optimal moment in order to successfully fertilise the cow. It is important to find the right moment to inseminate each individual animal in the farm, for efficiency reasons. In case the animal is not successfully inseminated, milk production is affected.

Several biomarker measurements may be made on the animal, such as e.g. measuring levels of progesterone, LDH (Lactate Dehydrogenase), BHB (Beta-Hydroxybutyrat) and urea. Thereby important information concerning e.g. heat detection and/or pregnancy of the individual animal may be made (based on measured progesterone level), as well as mastitis (based on LDH) and ketosis (based on BHB). Also, the energy balance may be estimated (based on urea).

A milk analysis apparatus/service module may be arranged to cooperate with a milk extracting arrangement, for regularly analysing milk samples of the animals, e.g. at or around the moment of a milking session. The milk analysis apparatus/service module may extract a milk sample and provide it on a dry stick/lateral flow stick/lateral flow test strip, or similar. The milk may be diluted with a diluent, which also may be used to rinse the tubings between test sessions. The optional diluent may be provided in a liquid container. Document WO 2018236271 shows an example of a milk analysis apparatus of this type. Thereby, a farmer/operator is provided with important information concerning each individual animal. The biomarker such as progesterone may be measured of all individual animals at the farm with a high degree of automation.

An emerging trend is to reduce width of lateral flow sticks in order to save material. Thereby, material is saved and the cost for the dry stick is reduced. 5 mm is often regarded as a prior art minimum recommended width of the dry stick. In case the width is more narrowly reduced, for example to 4 mm, 3.5 mm, 3 mm, 2.5 mm, 2 mm, etc., or when the lateral edge/s is/are incorrectly cut, for example several, problems may emerge.

A disadvantage is that the narrower width the dry stick has, the more challenging will it be to align the needle with the dry stick and apply the extracted milk sample onto the dry stick, i.e. to direct the needle to the sample pad of the dry stick. In case the milk sample is applied aside of the dry stick, the biomarker measurement will fail.

It would be desired to find a reliable way of applying the milk sample to a dry stick, also when the dry stick is very narrow in relation to the conventional width of about 5 mm.

### SUMMARY

It is therefore an object of this invention to solve at least some of the above problems and enable application of a milk sample of an animal to the dry stick also when the dry stick is very narrow, such as for example the dry stick has a width of about 1-3 mm, preferably 2-2.5 mm.

According to a first aspect of the invention, this objective is achieved by an arrangement arranged to align a needle and a dry stick in order to apply a milk sample of an animal to the dry stick when the dry stick is positioned in a milk sample apply position. The dry stick is configured to indicate at least one biomarker value of the applied milk sample.

A biomarker, or biological marker, generally refers to a measurable indicator of some biological state or condition of the animal. The biomarker value measurement may be associated with pregnancy/reproduction of the animal.

The arrangement comprises a carrier, comprising at least one dry stick. The carrier has a longitudinal extension, extending in a Y-direction. The carrier may for example be embodied as a carrier tape, on which the dry stick, or plurality of dry sticks are arranged. Alternatively, the carrier may be embodied as a blister package.

Also, the arrangement comprises a tube element arranged to receive the milk sample of the animal and the needle. The needle is connected to the tube element, wherein the needle is arranged to receive the milk sample of the animal via the tube element and apply the milk sample to the dry stick.

In addition, the arrangement also comprises a first light source directed to illuminate the dry stick for creating a first shadow of the dry stick on the carrier. The first light source may be embodied as a lamp, or flash.

The arrangement additionally comprises a camera configured to capture an image of at least a part of the dry stick and the first shadow of the part of the dry stick. Furthermore, the arrangement comprises a drive unit comprising an interface configured to cooperate with the carrier in order to move the dry stick on the carrier in the Y-direction to the milk sample apply position.

The arrangement also comprises a controller, communicatively connected to the camera and the drive unit. The controller is configured to trigger the camera to capture the image of the part of the dry stick and the first shadow of the part of the dry stick on the carrier, which first shadow has been created by the first light source. Additionally, the controller is configured to receive the captured image from the camera. The controller is also configured to perform an image analysis of the image, for detecting the part of the dry stick and the first shadow of the part of the dry stick on the carrier. The controller is finally configured to provide an apply milk signal to the drive unit to move the carrier and the dry stick in the Y-direction, into the milk sample apply position based on the image analysis, wherein the needle is enabled to apply the milk sample to the dry stick.

By creating a shadow of the dry stick on the carrier by illuminating the dry stick from the side with the first light source, a sharp contrast is created by the dry stick edge (which typically is white or very light) between the dry stick edge and the carrier. A correct location of the dry stick edge is thereby enabled by image analysis.

In a first implementation of the arrangement according to the first aspect, the controller may be configured to perform the image analysis of the image for detecting a first edge of the dry stick by detecting the first shadow of the first edge of the dry stick, on the carrier.

In a second implementation of the arrangement according to the first aspect, the needle is arranged at a predetermined position in the Y-direction and an X-direction which is perpendicular to the Y-direction. The predetermined position of the needle is known by the controller.

The image analysis of the image, performed by the controller may comprise detecting a section of a first edge of the dry stick by detecting the first shadow of the part of the dry stick on the carrier. The image analysis may also comprise extrapolating an extension of the section of the first edge of the dry stick at a segment of the dry stick where the milk sample is to be applied by the needle when the dry stick is in the milk sample apply position. In addition the image analysis may comprise calculating a distance in the Y-direction between the extrapolated extension of the first edge at the segment of the dry stick and the predetermined position of the needle. The image analysis may also comprise generating the milk apply signal to be provided to the drive unit to move the carrier and the dry stick in the Y-direction into the milk sample apply position, based on the calculated distance thereby aligning the predetermined position of the needle with the section of the dry stick where the milk sample is to be applied by the needle.

The controller is enabled to correct the positioning of the dry stick in the Y-direction, also when the camera is able to capture an image only of a part of the dry stick.

In a third implementation of the arrangement according to the first aspect, or any implementation thereof, the first light source may be directed to illuminate the dry stick at an angle of incidence of substantially 45±15 degrees.

The sharp and distinct first shadow of the first dry stick edge created on the carrier in this way by the first light source makes the first edge of the dry stick easily recognisable when running the image analysis.

In a fourth implementation of the arrangement according to the first aspect, or any implementation thereof, the controller may have knowledge of the width of the dry stick. The controller may also be configured to move the carrier and the dry stick in the Y-direction, for aligning the dry stick where the milk sample is to be applied, with the predetermined position of the needle, based on the image processing and with knowledge of the width of the dry stick.

Having determined the correct location of the first dry stick edge and with knowledge of the width of the dry stick, the controller can calculate an adjustment of the dry stick in the Y-direction, for bringing the dry stick into the milk sample apply position where the milk sample is applied via the needle in the middle between the two lateral edges of the dry stick.

In a fifth implementation of the arrangement according to the first aspect, or any implementation thereof, the arrangement may comprise a second light source. The second light source may be directed to illuminate the dry stick for creating a second shadow of a second edge of the dry stick on the carrier. The controller may also be configured to switch off the light of the first light source and ignite the second light source. In addition, the controller may be configured to trigger the camera to capture the image of the second edge of the dry stick and the second shadow of the second edge of the dry stick on the carrier. The controller may be configured to receive the captured image from the camera, and perform the image analysis of the image, for detecting the second edge of the dry stick by detecting the second shadow of the second edge of the dry stick, on the carrier.

The controller may also be configured to move the carrier and the dry stick in the Y-direction, for aligning the dry stick on which the milk sample is to be applied, with the predetermined position of the needle, based on the detected first edge of the dry stick and the second edge of the dry stick, respectively.

By correct positioning of the dry stick and an application of the milk sample symmetrically between the first and second lateral edges of the dry stick, a successful application of the milk sample to the dry stick is assured, reducing risks of applying the milk sample beside the dry stick, also when the dry stick is very narrow, such as for example about 1-3 mm.

In a sixth implementation of the arrangement according to the first aspect, or any implementation thereof, the dry stick has a thickness that extends in a Z-direction, which Z-direction is perpendicular to the Y-direction. The first shadow on the carrier may be created by light from the first light source and the part of the dry stick extending in the Z-direction.

The image analysis of the image may comprise detecting that two or more dry sticks have been stacked onto each other on the carrier in the Z-direction. The image analysis may then comprise the step of estimating extension of the first shadow of the part of the dry stick on the carrier in the Y-direction before the milk sample is applied to the dry stick. In addition the image analysis may comprise comparing the estimated shadow extension with an expected shadow extension value; and, when the estimated shadow extension exceeds the expected shadow extension value, generating a forward signal to be provided to the drive unit to forward the carrier in the Y-direction, to a subsequent dry stick on the carrier.

The dry sticks are rather thin and two dry sticks may sometimes by mistake be stacked one on top of the other on the carrier. The hereby provided quality-check could easily and automatically detect these manufacturing mistakes, and the carrier could be forwarded to the subsequent dry stick before the milk sample is applied.

In a seventh implementation of the arrangement according to the first aspect, or any implementation thereof, the dry stick may comprise a sample pad for receiving the milk sample. Also, the dry stick may comprise a conjugate pad comprising conjugate. The dry stick may in addition comprise an indication zone with a test line and a control line, arranged to indicate the biomarker of the milk sample, based on the conjugate. In addition the dry stick may also comprise an absorbent pad for absorbing the milk sample. The dry stick may also comprise a porous membrane for receiving a capillary flow of the milk sample from the sample pad via the conjugate pad and the indication zone to the absorbent pad, thereby forwarding conjugate dispersed into the milk sample to the indication zone.

The image captured by the camera may depict at least a part of the absorbent pad, and the shadow that is created by the part of the absorbent pad on the carrier when illuminated by the light source. The controller may be configured to detect unsuccessful flowing of the applied milk sample through the porous membrane to the absorbent pad of the dry stick. The detection of unsuccessful milk flow may be made by estimating an extension of the shadow of the absorbent pad in the Y-direction on the carrier, at a determined time period after having applied the milk sample. Also, the detection may comprise comparing the estimated shadow extension of the shadow of the absorbent pad on the carrier with an expected reference shadow value. The detection of unsuccessful milk flow may also comprise the step of discarding the dry stick when the estimated shadow extension of the shadow of the absorbent pad on the carrier is smaller than the expected reference shadow value.

Failed lateral flow tests could hereby be detected and one or several appropriate actions may be triggered, for example discarding the dry stick/test; generating an alert to the farmer; forwarding the carrier to a next dry stick for repeating the application of the milk sample to the new dry stick, etc.

Other advantages and additional novel features will become apparent from the subsequent detailed description.

### FIGURES

Embodiments of the invention will now be described in further detail with reference to the accompanying figures, in which:

FIG. 1 illustrates an example of an arrangement for measuring a biomarker value of a milk sample of an animal by aligning a needle and a dry stick in order to apply the milk sample of the animal to the dry stick.

FIG. 2A illustrates an example of a dry stick as regarded in a side view, according to an embodiment.

FIG. 2B illustrates an example of a dry stick as regarded from above, according to an embodiment.

FIG. 3A illustrates various components comprised in an arrangement for aligning a needle and a dry stick in order to apply a milk sample of an animal to the dry stick, assisted by a shadow of the dry stick on a carrier.

FIG. 3B illustrates an image of a dry stick, captured by a camera of the arrangement, according to an embodiment.

FIG. 4A illustrates a carrier comprising dry sticks, and a shadow on the carrier, created by light from a light source, according to an embodiment.

FIG. 4B illustrates an image of a part of a dry stick and a shadow thereof, captured by a camera of the arrangement, according to an embodiment.

FIG. 5 illustrates an image of a dry stick arranged on a carrier, and a shadow on the carrier, created by light from a light source, according to an embodiment.

FIG. 6A illustrates an image of a dry stick arranged on a carrier, and a shadow on the carrier, created by light from a light source, according to an embodiment.

FIG. 6B illustrates an image of a dry stick arranged on a carrier, and a shadow on the carrier, created by light from a light source, according to an embodiment.

### DETAILED DESCRIPTION

Embodiments of the invention described herein are defined as an arrangement which may be put into practice in the embodiments described below. These embodiments may, however, be exemplified and realised in many different forms and are not to be limited to the examples set forth herein; rather, these illustrative examples of embodiments are provided so that this disclosure will be thorough and complete.

Still other objects and features may become apparent from the following detailed description, considered in conjunction with the accompanying drawings. It is to be understood, however, that the drawings are designed solely for purposes of illustration and not as a definition of the limits of the herein disclosed embodiments, for which reference is to be made to the appended claims. Further, the drawings are not necessarily drawn to scale and, unless otherwise indicated, they are merely intended to conceptually illustrate the structures and procedures described herein.

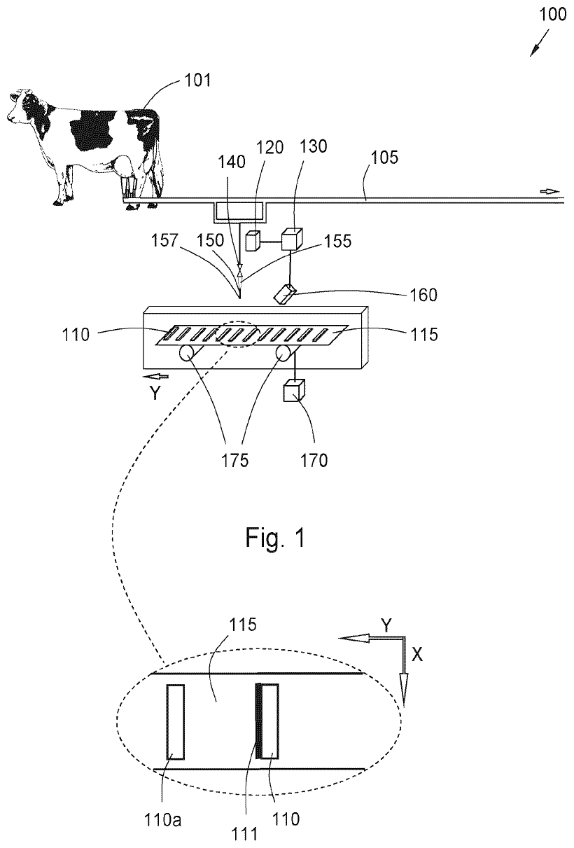

FIG. 1 illustrates a scenario with an arrangement 100 arranged to align a needle 150 and a dry stick 110 in order to apply a milk sample of an animal 101 to the dry stick 110 when the dry stick 110 is positioned in a milk sample apply position in which milk sample apply position the needle 150 is aligned with the dry stick 110.

“Animal” may be any arbitrary type of domesticated female milk producing and/or meat producing mammal such as cow, goat, sheep, horse, camel, dromedary, dairy buffalo, donkey, reindeer, yak, etc.

The lateral flow stick 110 may be individually arranged on a carrier 115 comprising a plurality of lateral flow sticks 110, 110a. Each one of the individually arranged lateral flow sticks 110, 110a on the carrier 115 may be arranged separated from each other.

The dry stick 110 is configured to indicate at least one biomarker value of the applied milk sample.

The carrier 115 has a longitudinal extension, extending in a Y-direction and a transverse extension, extending in an X-direction, wherein the Y-direction and the X-direction are perpendicular to each other.

Each one of the dry sticks 110, 110a has a longitudinal extension and may be arranged on the carrier 115 so that the longitudinal extension of the dry stick 110 extends on the carrier 115 along the X-direction with an inclination within a range of ±45 degrees in relation to the X-direction.

The carrier 115 may be embodied e.g. as a carrier tape, or a blister package. In embodiments wherein the carrier 115 is embodied as a carrier tape, the carrier 115 may be comprised in a cassette, for a swift handling.

Each dry stick 110, 110a may be maintained and sealed individually on the carrier 115 when embodied as a carrier tape, for example sealed by a foil covering the dry stick 110, 110a and the carrier 115. The foil may be made of transparent plastic/elastomere, for example. In these embodiments, the foil may be peeled off before the milk/liquid sample is applied to the dry stick 110, 110a. In case the carrier 115 is embodied as a blister package, the seal cover of the blister package may be punctured before application of the milk/liquid sample.

Milk of the animal 101 may be extracted and evacuated by a milking equipment that are part of a milking parlour via a milk line to a milk tank or similar milk storage.

During milking of the animal 101, or in close relation therewith, the milk sample may be diverted from the milking equipment/milk line 105 and provided to a tube element 155, having an interface to the milking equipment/milk line 105. A pump may be configured to act on the tube element 155, to forward the milk sample, from the milking equipment/milk line 105, via a valve 140 and a mixing chamber, to a needle 150.

In some embodiments, the milk sample may be mixed with a diluent in the mixing chamber, thereby forming a liquid sample comprising a mixture of milk and diluent.

The needle 150 is connected to the tube element 155, possibly via the valve 140 and the mixing chamber. The needle 150 is arranged to receive the milk sample of the animal 101 and apply the milk sample to the dry stick 110.

The needle 150 may be arranged at a predetermined position in the Y-direction and in the X-direction.

The arrangement 100 also comprises a first light source 160, directed to illuminate the dry stick 110 for creating a first shadow 111 of the dry stick 110 on the carrier 115.

The first light source 160 may be directed to illuminate the dry stick 110 at an angle of incidence of substantially 45±15 degrees. The first light source 160 may comprise a lamp arranged with a predetermined angle of incidence, with a predetermined illuminance. Illuminance is the total luminous flux incident on a surface, per unit area. The first light source 160 may thereby be applied to ensure proper illumination of the dry stick 110 and create a distinguished first shadow 111 of the dry stick 110 on the carrier 115.

The arrangement 100 in addition comprises a camera 120 configured to capture an image of at least a part of the dry stick 110 and the first shadow 111 of the part of the dry stick 110 on the carrier 115.

The arrangement 100 also comprises a drive unit 170 comprising an interface 175 configured to cooperate with the carrier 115 in order to move the dry stick 110 on the carrier 115 in the Y-direction to the milk sample apply position.

The drive unit 170 may comprise a motor such as a linear motor or engine such as an electric engine in some embodiments.

In case the carrier 115 is embodied as a carrier tape, the carrier tape may be arranged on a first spool and a second spool, possibly on a cassette. The interface 175 may then comprise one or two engaging wheels, arranged to rotate the first and/or the second spool, thereby causing the carrier 115 to move.

The arrangement 100 also comprises a controller 130, communicatively connected to the camera 120 and the drive unit 170, possibly also to the first light source 160.

The controller 130 is configured to perform a number of actions for aligning the needle 150 and the dry stick 110. The controller 130 is configured to trigger the camera 120 to capture an image of the part of the dry stick 110 and the first shadow 111 of the part of the dry stick 110 on the carrier 115. The first shadow 111 is created by the light of the first light source 160. The controller 130 is also configured to receive the captured image from the camera 120. The controller 130 is also configured to perform an image analysis of the image, for detecting the part of the dry stick 110 and the first shadow 111 of the part of the dry stick 110 on the carrier 115.

The first shadow 111 is created on the carrier 115, thereby creating a sharp and easily recognisable contrast, enabling recognition of a part of the dry stick 110 where the milk sample may be applied via the needle 150 by the image analysis.

Based on the image analysis, the controller 130 is configured to provide an apply milk signal to the drive unit 170 to move the carrier 115 and the dry stick 110 in the Y-direction via the interface 175, into the milk sample apply position, wherein the needle 150 is enabled to apply the milk sample to the dry stick 110.

The needle 150 may be arranged at a predetermined position in the Y-direction and in the X-direction, which predetermined position is known by the controller 130. The controller 130 is thereby enabled to align the needle 150 and the dry stick 110 and apply the milk sample to the dry stick 110 via the needle 150 when the dry stick 110 is positioned in the milk sample apply position.

The dry stick 110 may react on presence and/or amount of one or several biomarkers, e.g. by changing colours, colour tint or intensity of a colour/tint. The camera 120 may capture an image of the dry stick 110. The captured image of the dry stick 110 may then be analysed by the controller 130, and based on the intensity of the colour, presence and/or quantity of the biomarker in the milk sample may be estimated.

The measured biomarker may be e.g. progesterone, glycoprotein, oestrogen and/or Gonadatropin-Releasing Hormones, or any other similar biomarker associated with reproduction of the animal 101, in different embodiments.

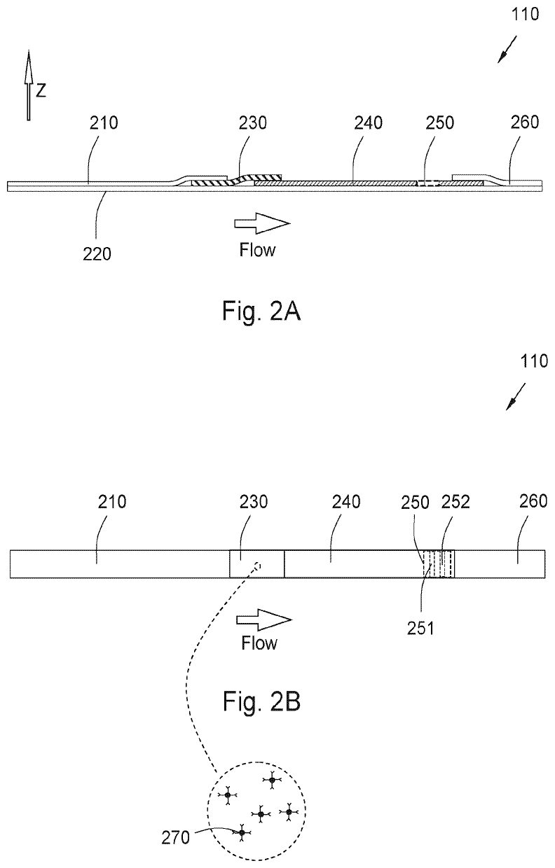

FIG. 2A illustrates a dry stick 110 as regarded from a side view while FIG. 2B illustrates the dry stick 110 as regarded from above.

The dry stick 110 may comprise a back plate 220, which may be made of cardboard, paper or similar material. The back plate 220 may form a base on to which other components of the dry stick 110 may be mounted.

The dry stick 110 may comprise a sample pad 210. The sample pad 210 is the part of the dry stick 110 onto which the milk sample/liquid sample is applied by the needle 150 when the dry stick 110 is situated in the milk sample apply position. The sample pad 210 may comprise a porous structure for enabling capillary flow such as cellulose fibres and/or woven meshes.

The dry stick 110 may also comprise a conjugate pad 230 comprising conjugate 270. The conjugate 270 may comprise antibody treated gold particles which are dispersed into the milk when the milk sample/liquid sample flows from the sample pad 310 through the conjugate pad 230.

The conjugate 270 will react with the biomarker to be measured, for example progesterone in the milk of the applied liquid sample.

The conjugate 270 may thus be embedded in the conjugate pad 230 which may comprise a glass-fibre section of the dry stick 110. Alternatively, the conjugate pad 230 may comprise cellulose and/or surface modified polyester.

Also, the dry stick 110 may comprise an indication zone 250, arranged to indicate the biomarker of the milk sample/liquid sample, based on the conjugate 270. The dry stick 110 furthermore may comprise a porous membrane 240 for receiving a capillary flow of the milk sample/liquid sample from the conjugate pad 230, thereby forwarding conjugate 270 dispersed into the milk sample/liquid sample to the indication zone 250.

The porous membrane 240 may comprise for example a nitrocellulose membrane, cellulose, glass fibre, polyester, rayon, a polymer, glass fibre, woven fibres, non-woven fibres, a chromatographic gel membrane, diatomaceous earth, silica gel, silicium oxide, kieselguhr, or other filtration membranes in different embodiments. The porous membrane 240 may be designed to enhance capillary pumping speed of liquid through the dry stick 110. The indication zone 250 of the porous membrane 240 may comprise a test line 251 and a control line 252.

The test line 251 may be treated with a biomarker reference such as e.g. a progesterone reference which binds the antibody treated gold particles of the milk sample/liquid sample and thereby brings the test line 251 to change colour tint when exposed for milk comprising a progesterone level lower than a threshold limit. Thus, the test line 251 may change colour tint into red/reddish when the milk has no or low progesterone level. If/when the animal 101 is in heat, the progesterone level is near zero in the milk sample. This colour change may then be detected by the camera 120 and may be reported to the farmer and/or stored in a database associated with an identity of the animal 101, and/or a time stamp. This may also trigger scheduling of insemination of the animal 101 (in case the biomarker is progesterone), or the scheduling of a veterinary check-up, for example (in case of another biomarker).

The control line 252 in the indication zone 250 of the porous membrane 240 may be treated with an antibody reference which binds antibody reference treated gold particles of the milk sample/liquid sample regardless of the progesterone level in the milk, and may thereby bring the control line 252 to change colour tint when exposed for milk comprising antibody reference treated gold particles, assuring a successful capillar flow of the milk/liquid through the porous membrane 240.

The dry stick 110 may comprise an absorbent pad 260. The absorbent pad 260 may comprise an absorbent configured to absorb superfluous milk/liquid from the porous membrane 240.

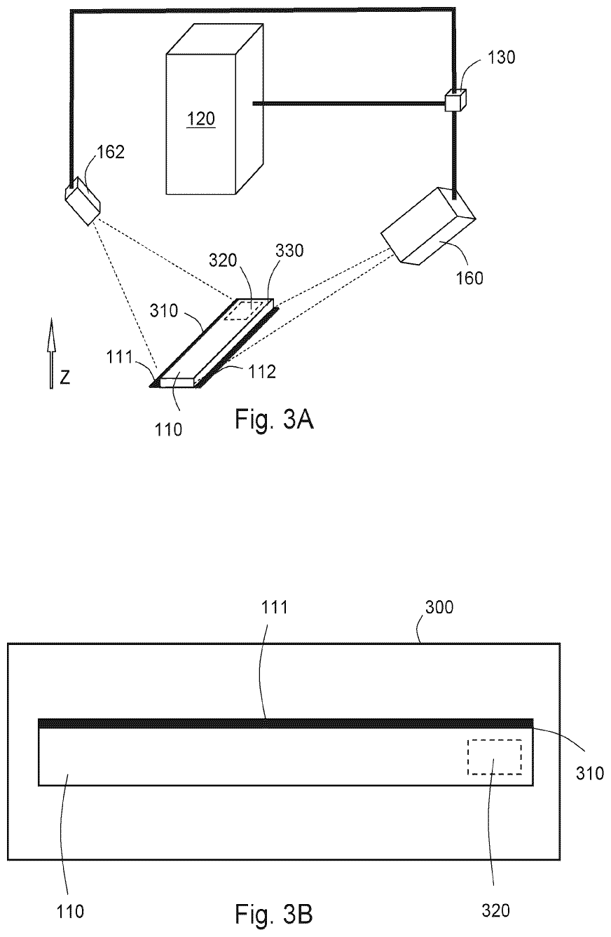

FIG. 3A schematically illustrates some elements comprised in the arrangement 100, such as the camera 120, the controller 130, and the first light source 160 illuminating the dry stick 110.

The dry stick 110 has a thickness that extends in a Z-direction. The Z-direction is perpendicular to the Y-direction and/or the X-direction. The first shadow 111 of the part of the dry stick 110 on the carrier 115 is created by light from the first light source 160 and a first edge 310 of the part of the dry stick 110 extending in the Z-direction.

The controller 130 may be configured to perform the image analysis of the image by a number of actions.

The controller 130 may be configured to detect a section of the first edge 310 of the dry stick 110 by detecting the first shadow 111 of the first edge 310 of the dry stick 110 extending in the Z-direction on the carrier 115.

In some embodiments, the arrangement 100 may comprise a second light source 162 directed to illuminate the dry stick 110 so that a second edge 330 of the dry stick 110 creates a second shadow 112 of the dry stick 110 on the carrier 115, while the first light source 160 is switched off. The second light source 162 may then illuminate the dry stick 110 from an opposite side of the dry stick 110 in relation to the first light source 160.

The controller 130 is thereby enabled to detect both the lateral edges 310, 330 of the dry stick 110 and is thereby enabled to apply the milk sample via the needle 150 to the segment 320 of the dry stick 110 where the milk sample is to be applied in between the first and the second lateral edges 310, 330 of the dry stick 110, also when the controller 130 does not know the width of the dry stick 110, or when different dry sticks 110 have different widths.

The second light source 162 may be directed to illuminate the dry stick 110 at an angle of incidence of substantially 45±15 degrees, similar to the first light source 160, but from the opposite lateral side of the dry stick 110.

FIG. 3B illustrates an image 300 of the dry stick 110, and the first shadow 111 of the first edge 310 captured by the camera 120.

The sharp and distinguished first shadow 111 created by the first edge 310 of the dry stick 110 on the carrier 115, makes it easy for the controller 130 to determine position of the edge 310, which in turn enable precision when aligning the needle 150 with the segment 320 of the dry stick 110 where the milk sample is to be applied by the needle 150 when the dry stick 110 is in the milk sample apply position, also when the dry stick 110 is very narrow, such as in an interval of about 1-3 mm, preferable 2-2.5 mm, or there about.

The controller 130 may have knowledge of the width of the dry stick 110, for example 2 mm. The width of the dry stick 110 may be manually entered by the operator or estimated by image analysis of the image 300 captured by the camera 120. Having determined the correct location of the dry stick edge 310 and with knowledge of the width of the dry stick 110, the controller 130 can move the carrier 115 and the dry stick 110 in the Y-direction, for aligning the segment 320 of the dry stick 110 where the milk sample is to be applied, with the predetermined position of the needle 150, based on the image processing and with knowledge of the width of the dry stick 110. Thereafter, the controller 130 may apply the milk sample via the needle 150 at a distance corresponding to the width of the dry stick 110 divided by 2, from the first dry stick edge 310.

However, the controller 130 may also apply the herein described methodology on a dry stick 110, having a width of at least 5 mm. Hereby, enhanced successfulness concerning application of the milk sample to the dry stick 110 is achieved.

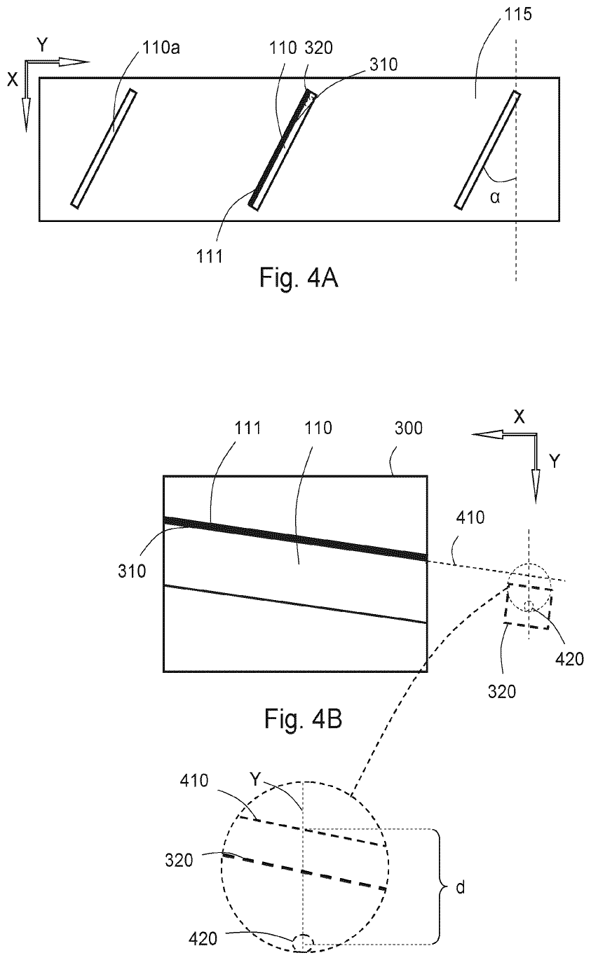

FIG. 4A illustrates an example of the carrier 115 as regarded from above. The dry sticks 110, 110a on the carrier 115 are arranged with an inclination a within a range of ±45 degrees in relation to the X-direction (non-limiting example).

FIG. 4B illustrates an example of an image 300 captured by the camera 120.

In this example, only a part of the dry stick 110 is captured in the image 300.

The controller 130 may be configured to detect a section of a first edge 310 of the dry stick 110 in the image 300, by detecting the first shadow 111 on the carrier 115, of the part of the dry stick 110 extending in the Z-direction.

The controller 130 may be configured to extrapolate an extension 410 of the first edge 310 of the section of the dry stick 110 at a segment 320 of the dry stick 110 where the milk sample is to be applied by the needle 150 when the dry stick 110 is in the milk sample apply position.

Then, the controller 130 may calculate a distance d in the Y-direction between the extrapolated extension 410 of the first edge 310 and a predetermined position 420 in the Y-direction and the X-direction, of the needle 150.

The controller 130 may also be configured to generate the milk apply signal to be provided to the drive unit 170 to move the carrier 115 and the dry stick 110 in the Y-direction into the milk sample apply position. The milk apply signal and the size of the movement in the Y-direction may be based on the calculated distance d. Thereby, the predetermined position 420 of the needle 150 in the Y-direction and the X-direction is aligned with the segment 320 of the dry stick 110 where the milk sample is to be applied by the needle 150.

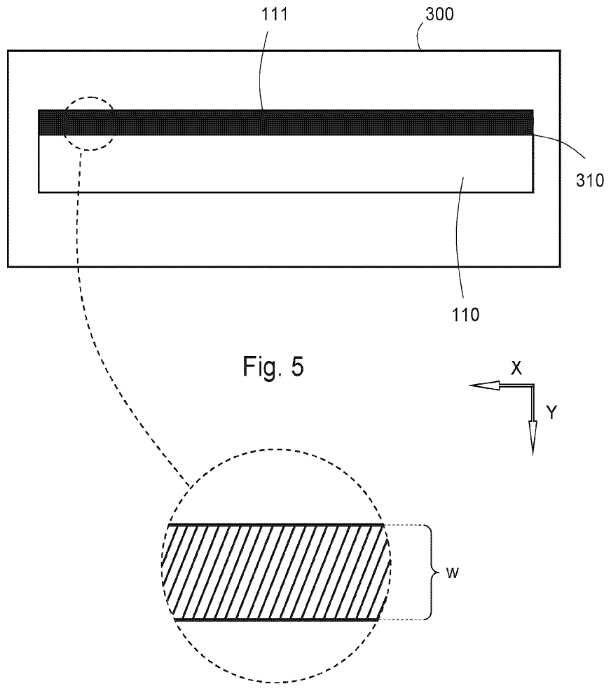

FIG. 5 illustrates an example of an image 300 captured by the camera 120, depicting the dry stick 110.

A first edge 310 of the dry stick 110 creates a first shadow 111 on the carrier 115. The first shadow 111 enable a very precise positioning of the first edge 310 by the controller 130. The first shadow 111 created by the dry stick 110 has an extension w in the Y-direction.

The image analysis may in some embodiments comprise detecting that two or more dry sticks 110 by mistake have been stacked onto each other on the carrier 115, in the Z-direction. The controller 130 may be configured to estimate extension w of the shadow 111 on the carrier 115, of the part of the dry stick 110 in the Y-direction before the milk sample is applied to the dry stick 110.

The controller 130 may also be configured to compare the estimated shadow extension w on the carrier 115 with an expected shadow extension value. The expected shadow extension value typically corresponds with the shadow extension w of the shadow 111 of a single dry stick 110 on the carrier 115. When the estimated shadow extension w exceeds the expected shadow extension value, it may at least implicitly be concluded that two or more dry sticks 110 have been stacked onto each other; or possibly that some other non-desired object has accidently been placed on/under the dry stick 110. In any way, it is inappropriate to use the dry stick 110 for making tests such as lateral flow tests.

The controller 130 may also be configured to generate a forward signal to be provided to the drive unit 170 to forward the carrier 115 in the Y-direction, to a subsequent dry stick 110a on the carrier 115.

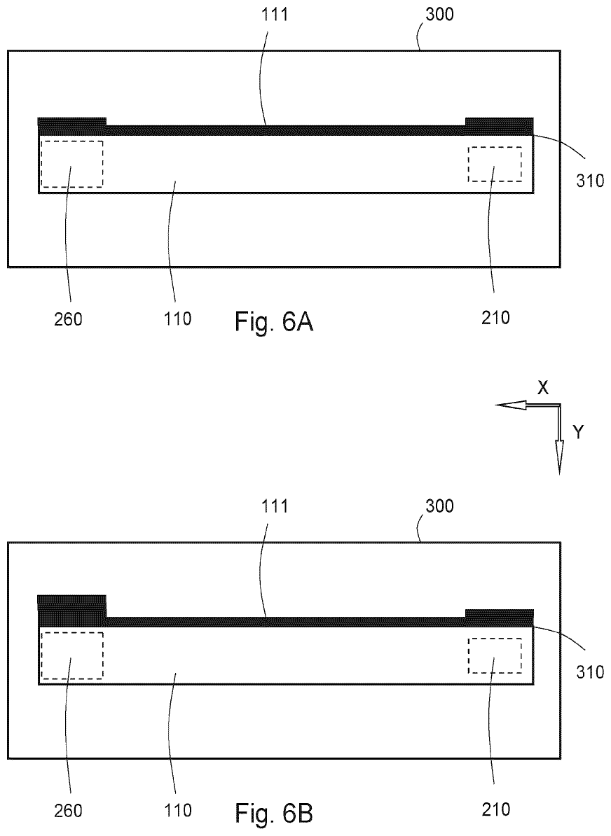

FIG. 6A illustrates an image 300 depicting a dry stick 110 comprising a sample pad 210 for receiving the milk sample via the needle 150, before application of a milk sample. The illustrated dry stick 110 also comprises an absorbent pad 260, for absorbing the milk sample.

The dry stick 110 may for example comprise a lateral flow stick as illustrated in FIGS. 2A-2B and discussed in the corresponding section of the specification.

FIG. 6B illustrates an image 300 depicting the dry stick 110 and a shadow 111 of the dry stick 110 on the carrier 115, after application of the milk sample to the sample pad 210, and after the milk sample having propagated by a capillary flow from the sample pad 210 via the conjugate pad 230 and the indication zone 250 to the absorbent pad 260 of the dry stick 110.

The controller 130 may in some embodiments be configured to detect unsuccessful flowing of the applied milk sample through the porous membrane 240 to the absorbent pad 260 of the dry stick 110.

The detection of unsuccessful flowing may comprise estimating an extension w of the shadow 111 on the carrier 115, of the absorbent pad 260 in the Y-direction, at a determined time period after having applied the milk sample.

The determined time period may correspond with an estimated propagation of milk flowing through the dry stick 110 to the absorbent pad 260, after having applied the milk sample to the sample pad 210.

Also, the controller 130 may be configured to compare the estimated shadow extension w of the shadow 111 on the carrier 115, of the absorbent pad 260, with an expected reference shadow value.

The reference shadow value may approximately correspond with a shadow extension of the shadow 111 of the absorbent pad 260 when milk/liquid has been absorbed by the absorbent pad 260, after the determined time period.

The controller 130 may be configured to discard the dry stick 110 when the estimated shadow extension w of the shadow 111 on the carrier 115, of the absorbent pad 260 is smaller than the expected reference shadow value.

The controller 130 may be embodied as a processing circuitry configured for performing various calculations for conducting various computer programs, for example to communicate with the camera 120 and/or the drive unit 170, respectively, and/or to perform the image analysis.

The processing circuitry may comprise one or more instances of a processing circuit, i.e. a Central Processing Unit (CPU), a processing unit, a processing circuit, a processor, an Application Specific Integrated Circuit (ASIC), a microprocessor, or other processing logic that may interpret and execute instructions. The herein utilised expression “processing circuitry” may thus represent a plurality of interconnected and cooperating processing circuits, such as, e.g., any, some or all of the ones enumerated above.

Furthermore, the controller 130 may comprise a memory in some embodiments. The optional memory may comprise a physical device utilised to store data or programs, i.e., sequences of instructions, on a temporary or permanent basis. According to some embodiments, the memory may comprise integrated circuits comprising silicon-based transistors. The memory may comprise e.g. a memory card, a flash memory, a USB memory, a hard disc, or another similar volatile or non-volatile storage unit for storing data such as e.g. ROM (Read-Only Memory), PROM (Programmable Read-Only Memory), EPROM (Erasable PROM), EEPROM (Electrically Erasable PROM), etc. in different embodiments.

Further, the controller 130 may comprise a signal transmitter. The signal transmitter may be configured for transmitting signals via a wired or wireless communication interface to the camera 120, the drive unit 170 and/or the needle 150 possibly via a transceiver; and/or to the database.

Furthermore, a computer program comprising instructions to perform the image analysis of the image 300 captured by the camera 120 may be performed in the controller 130.

The computer program mentioned above may be provided for instance in the form of a computer-readable medium, i.e. a data carrier carrying computer program code for performing at least some of the computer program steps, according to some embodiments when being loaded into the one or more processing circuitries of the controller 130. The data carrier may be, e.g., a hard disk, a CD ROM disc, a memory stick, an optical storage device, a magnetic storage device or any other appropriate medium such as a disk or tape that may hold machine readable data in a non-transitory manner. The computer program may furthermore be provided as computer program code on a server and downloaded to the controller 130 remotely, e.g. over an Internet or an intranet connection.

The embodiments, or parts thereof, illustrated in FIG. 1, FIG. 2A, FIG. 2B, FIG. 3A, FIG. 3B, FIG. 4A, FIG. 4B, FIG. 5, FIG. 6A and/or FIG. 6B may with advantage be combined with each other for achieving further benefits.

The terminology used in the description of the embodiments as illustrated in the accompanying drawings is not intended to be limiting of the described arrangement 100. Various changes, substitutions and/or alterations may be made, without departing from invention embodiments as defined by the appended claims.

As used herein, the term “and/or” comprises any and all combinations of one or more of the associated listed items. The term “or” as used herein, is to be interpreted as a mathematical OR, i.e., as an inclusive disjunction; not as a mathematical exclusive OR (XOR), unless expressly stated otherwise. In addition, the singular forms “a”, “an” and “the” are to be interpreted as “at least one”, thus also possibly comprising a plurality of entities of the same kind, unless expressly stated otherwise. It will be further understood that the terms “includes”, “comprises”, “including” and/or “comprising”, specifies the presence of stated features, actions, integers, steps, operations, elements, and/or components, but do not preclude the presence or addition of one or more other features, actions, integers, steps, operations, elements, components, and/or groups thereof. A single unit such as e.g. a processor may fulfil the functions of several items recited in the claims. The mere fact that certain measures or features are recited in mutually different dependent claims, illustrated in different figures or discussed in conjunction with different embodiments does not indicate that a combination of these measures or features cannot be used to advantage. A computer program may be stored/distributed on a suitable medium, such as an optical storage medium or a solid-state medium supplied together with or as part of other hardware, but may also be distributed in other forms such as via Internet or other wired or wireless communication system.

1. An arrangement configured to align a needle and a dry stick in order to apply a milk sample of an animal to the dry stick when the dry stick is positioned in a milk sample apply position in which the needle is aligned with the dry stick, wherein the dry stick is configured to indicate at least one biomarker value of the applied milk sample; and wherein the arrangement comprises:
a carrier comprising at least one dry stick; wherein the carrier has a longitudinal extension, extending in a Y-direction;
a tube element configured to receive the milk sample of the animal;
a needle connected to the tube element, wherein the needle is configured to receive the milk sample of the animal via the tube element and apply the milk sample to the dry stick;
a first light source directed to illuminate the dry stick for creating a first shadow of the dry stick on the carrier;
a camera configured to capture an image of at least a part of the dry stick and the first shadow of the part of the dry stick on the carrier;
a drive unit comprising an interface configured to cooperate with the carrier in order to move the dry stick on the carrier in the Y-direction to the milk sample apply position;
a controller, communicatively connected to the camera and the drive unit, wherein the controller is configured to
trigger the camera to capture the image of the part of the dry stick and the first shadow of the part of the dry stick on the carrier;
receive the captured image from the camera;
perform an image analysis of the image, for detecting the part of the dry stick and the first shadow of the part of the dry stick on the carrier;
provide an apply milk signal to the drive unit to move the carrier and the dry stick in the Y-direction, into the milk sample apply position based on the image analysis, wherein the needle is enabled to apply the milk sample to the dry stick.

2. The arrangement according to claim 1, wherein the controller is configured to perform the image analysis of the image for detecting a first edge of the dry stick by detecting the first shadow of the first edge of the dry stick, on the carrier.

3. The arrangement according to claim 1, wherein the needle is configured at a predetermined position in the Y-direction and an X-direction perpendicular to the Y-direction, wherein the predetermined position is known by the controller; and wherein the image analysis of the image, performed by the controller comprises
detecting a section of a first edge of the dry stick by detecting the first shadow of the part of the dry stick on the carrier;
extrapolating an extension of the section of the first edge of the dry stick at a segment of the dry stick where the milk sample is to be applied by the needle when the dry stick is in the milk sample apply position; and
calculating a distance (d) in the Y-direction between the extrapolated extension of the first edge at the segment of the dry stick and the predetermined position of the needle; and
generating the milk apply signal to be provided to the drive unit to move the carrier and the dry stick in the Y-direction into the milk sample apply position, based on the calculated distance (d) thereby aligning the predetermined position of the needle with the segment of the dry stick where the milk sample is to be applied by the needle.

4. The arrangement according to claim 1, wherein the first light source is directed to illuminate the dry stick at an angle of incidence of substantially 45±15 degrees.

5. The arrangement according to claim 1, wherein the controller have knowledge of the width of the dry stick and is configured to move the carrier and the dry stick in the Y-direction, for aligning the dry stick on which the milk sample is to be applied, with the predetermined position of the needle, based on the image processing and with knowledge of the width of the dry stick.

6. The arrangement according to claim 2, comprising a second light source directed to illuminate the dry stick for creating a second shadow of a second edge of the dry stick on the carrier; and wherein the controller is configured to
switch off the light of the first light source;
ignite the second light source;
trigger the camera to capture the image of the second edge of the dry stick and the second shadow of the second edge of the dry stick on the carrier;
receive the captured image from the camera; and
perform the image analysis of the image, for detecting the second edge of the dry stick by detecting the second shadow of the second edge of the dry stick, on the carrier;
and wherein the controller is configured to move the carrier and the dry stick in the Y-direction, for aligning the dry stick on which the milk sample is to be applied, with the predetermined position of the needle, based on the detected first edge of the dry stick and the second edge of the dry stick, respectively.

7. The arrangement according to claim 1, wherein the dry stick has a thickness that extends in a Z-direction, which Z-direction is perpendicular to the Y-direction; and wherein the first shadow on the carrier is created by light from the first light source and the part of the dry stick extending in the Z-direction; and wherein the image analysis of the image comprises detecting that two or more dry sticks have been stacked onto each other on the carrier in the Z-direction, by:
estimating extension (w) of the first shadow of the part of the dry stick on the carrier in the Y-direction before the milk sample is applied to the dry stick;
comparing the estimated shadow extension (w) with an expected shadow extension value; and, when the estimated shadow extension (w) exceeds the expected shadow extension value,
generating a forward signal to be provided to the drive unit to forward the carrier in the Y-direction, to a subsequent dry stick on the carrier.

8. The arrangement according to claim 1, wherein the dry stick comprises:
a sample pad for receiving the milk sample;
a conjugate pad comprising conjugate;
an indication zone with a test line and a control line, configured to indicate the biomarker of the milk sample, based on the conjugate;
an absorbent pad for absorbing the milk sample; and
a porous membrane for receiving a capillary flow of the milk sample from the sample pad via the conjugate pad and the indication zone to the absorbent pad, thereby forwarding conjugate dispersed into the milk sample to the indication zone; and wherein
the image captured by the camera depicts at least a part of the absorbent pad, and the first shadow of the part of the absorbent pad on the carrier;
the controller is configured to detect unsuccessful flowing of the applied milk sample through the porous membrane to the absorbent pad of the dry stick, by:
estimating an extension (w) of the first shadow of the absorbent pad on the carrier in the Y-direction, at a determined time period after having applied the milk sample;
comparing the estimated shadow extension (w) of the first shadow of the absorbent pad on the carrier with an expected reference shadow value; and
discarding the dry stick when the estimated shadow extension (w) of the first shadow of the absorbent pad on the carrier is smaller than the expected reference shadow value.
